SEQUENCE LISTING (3 sequences)

SEQ ID NO: 1 - DNA, 2790 bp, Magnaporthe grisea
atggcgtatagaggtgctggagggccgggaggcggccgtgactacgatggtcacaacatgcaagacctaaaccctcacagccagtattccaatgtccaattgcctcctgggcacgagcaagaggacgaggcacacaggtcccttttgacccaaggcacaacgctctacgaccacgaccgactcggcgcccacacgcctccagtgcgtccagtctcggcctacagtctcacagagtcctatgccccgaatgctcccaccaccgttcctggctcggccgtcggcgcatctccctcgccatttcagaatgactatggtgtttcgtcgggctaccagggcgcaatgggcggccacgcagacgacggcttccccattggcggcggagacccccagcaaggccacccctacgataccgaggacagctgggtgcagcgccagaaccccaacgccgctccccagggtggtggtctgaaacgttatgccaccaggaaggtcaagctggtgcagggttcagtcctgagcattgactacaacgttcccagtgccatcaggaacgccgtgcagcccaagtaccgcgagcaggagggcaccaacgaggagtttatcaagatgcgatacaccgcggccacctgtgaccccaacgacttcaccctgaagaacggttacgatttgcgtccccgcatgtataacaggcacacggagcttctgatcgccatcacctactacaacgaggacaaggtactgctgtcccgtaccctgcacggtgttatgcagaacattagggacatcgtgaacctgaagaagtcgactttctggaacaaaggcggcccggcttggcagaagattgttgtctgcttggttttcgacggtatcgagaagaccgacaagaacgttctggacgtcctcgccaccattggtatctaccaagatggcgtcgtcaagaaggacgttcacggccaggagactgttgctcatattttcgagtacaccactcagctgtccgtcaccccgagccagcagctcatccgaccccaggacgacggccccaacactctgccgcctgtccagttcatcttctgtctcaaggccaagaatagcaagaagatcaactctcaccgttggcttttcaacgcctttggacggatcctgaaccccgaggtctgtattctgctggatgccggtaccaagcccagctcgaggtcgcttctcggtctctgggagggtttttacaacgacaaggatcttggaggtgcttgcggtgaaattcatgctatgcttggaaagggcggccggaagctgctcaaccccctcgtagctgtgcagaacttcgagtacaagatctccaacattctcgacaagcctctggagagtgcgttcggatacgttagtgtgttgcccggtgctttctccgcataccgcttccgtgccatcatgggacgacccttggagcaatacttccacggtgaccacaccctgtccaagattctgggcaagaagggtatcgagggcatgaacattttcaagaagaacatgttcttggccgaagatcgtattctttgtttcgagcttgttgccaaggccggccagaaatggcacctgagctacatcaaagctgccaagggagagaccgatgttcctgagggtgctgccgagttcatcagccagcgtcgtcgttggttgaacggttcgtttgccgccaccttgtactcactgatgcacttcggaaggatgtacaagagtggtcataacatcatccgcatgttcttcttccacgtccagctcatctacaacatcctcaacgtcatcttcacctggttctctctggcatcttactggcttaccaccaccgtcatcatggatctcgtcggaaacccccaagttggacagaacgctcgcgaaggctggccttttggtaacaccgtcacgccactcttcaacgccgttctcaagtacatttacctggcctttgtcatcctacagtttattctggccttgggtaatagaccgaagggatccaagtacacctacgtcacgtcattctttgtcttctcggtcatccaggcgtatatcctggttttgtctggatacctcgtcgtccaggccttccagacacctatcggcgagcagatcaagaccgacacggccaaggatttcatggatagtatatttggaaagagcggagccgctggtgttattctactcgccttgatcgccatctacggtatttacttcatcgcctcgttcatgtacctggacccctggcacatgttccactccttcccttactacatgctgctcatgtccacctacatcaacattctcatggtgtacgccttcaacaactggcacgatgtttcttggggtaccaaaggatccgacagtaatgaagctctaccctcggccaatattaccaaaggcgagaaggacgaagtcgtcgtggaggaaatcgacaagcctcaagaggatatcgacagtcagttcgaagcaactgtccgtcgtgccctggcacctttcaacgacgaggagaagcccgagcccaaggatctcgaggactcgtacaagtctttccgtacaatgctggttgttctctggctcttctccaactgtctcttggctgttgcgattactagcgataatttcgacgctttgacaaagaaccaaaacacggccaggactgcctctttcttccagttcttgctgttctcgaccgctttcctctctttgatccgtttcatcggattcctctggttcttgggcaagaccggtataatgtgctgcattgcccgtcgctaa

SEQ ID NO: 2 - DNA, 3346 bp, Magnaporthe grisea
ccgctgaagccgccttgcctgcaattttttctgctacttaccgcactcattagcgggagtcggcgctctttgttctccatccatttccgggtttccagatcagggtacggcggaaaacccaataacgtctgctctgttgtgtcaccacttttttccaccttctagtttccactcgcacgctcgttacccgctgagatacccaagatcatcacatcgcaatggcgtatagaggtgctggagggccgggaggcggccgtgactacgatggtcacaacatgcaagacctaaaccctcacagccaggtaggaagccatcgcgactaatctccccagcaaagtttgctcaacttgcaaacagatggagtttgccgtggacgacgctgatttcaaatatcttctgcacacagtattccaatgtccaattgcctcctgggcacgagcaagaggacgaggcacacaggtcccttttgacccaaggcacaacgctctacgaccacgaccgactcggcgcccacacgcctccagtgcgtccagtctcggcctacagtctcacagagtcctatgccccgaatgctcccaccaccgttcctggctcggccgtcggcgcatctccctcgccatttcagaatgactatggtgtttcgtcgggctaccagggcgcaatgggcggccacgcagacgacggcttccccattggcggcggagacccccagcaaggccacccctacgataccgaggacagctgggtgcagcgccagaaccccaacgccgctccccagggtggtggtctgaaacgttatgccaccaggaaggtcaagctggtgcagggttcagtcctgagcattgactacaacgttcccagtgccatcaggaacgccgtgcagcccaagtaccgcgagcaggagggcaccaacgaggagtttatcaagatgcgatacaccgcggccacctgtgaccccaacgacttcaccctgaagaacggttacgatttgcgtccccgcatgtataacaggcacacggagcttctgatcgccatcacctactacaacgaggacaaggtactgctgtcccgtaccctgcacggtgttatgcagaacattagggacatcgtgaacctgaagaagtcgactttctggaacaaaggcggcccggcttggcagaagattgttgtctgcttggttttcgacggtatcgagaagaccgacaagaacgttctggacgtcctcgccaccattggtatctaccaagatggcgtcgtcaagaaggacgttcacggccaggagactgttgctcatattttcgagtacaccactcagctgtccgtcaccccgagccagcagctcatccgaccccaggacgacggccccaacactctgccgcctgtccagttcatcttctgtctcaaggccaagaatagcaagaagatcaactctcaccgttggcttttcaacgcctttggacggatcctgaaccccgaggtctgtattctgctggatgccggtaccaagcccagctcgaggtcgcttctcggtctctgggagggtttttacaacgacaaggatcttggaggtgcttgcggtgaaattcatgctatgcttggaaagggcggccggaagctgctcaaccccctcgtagctgtgcagaacttcgagtacaagatctccaacattctcgacaagcctctggagagtgcgttcggatacgttagtgtgttgcccggtgctttctccgcataccgcttccgtgccatcatgggacgacccttggagcaatacttccacggtgaccacaccctgtccaagattctgggcaagaagggtatcgagggcatgaacattttcaagaagaacatgttcttggccgaagatcgtattctttgtttcgagcttgttgccaaggccggccagaaatggcacctgagctacatcaaagctgccaagggagagaccgatgttcctgagggtgctgccgagttcatcagccagcgtcgtcgttggttgaacggttcgtttgccgccaccttgtactcactgatgcacttcggaaggatgtacaagagtggtcataacatcatccgcatgttcttcttccacgtccagctcatctacaacatcctcaacgtcatcttcacctggttctctctggcatcttactggcttaccaccaccgtcatcatggatctcgtcggaaacccccaagttggacagaacgctcgcgaaggctggccttttggtaacaccgtcacgccactcttcaacgccgttctcaagtacatttacctggcctttgtcatcctacagtttattctggccttgggtaatagaccgaagggatccaagtacacctacgtcacgtcattctttgtcttctcggtcatccaggcgtatatcctggttttgtctggatacctcgtcgtccaggccttccagacacctatcggcgagcagatcaagaccgacacggccaaggatttcatggatagtatatttggaaagagcggagccgctggtgttattctactcgccttgatcgccatctacggtatttacttcatcgcctcgttcatgtacctggacccctggcacatgttccactccttcccttactacatgctgctcatgtccacctacatcaacattctcatggtgtacgccttcaacaactggcacgatgtttcttggggtaccaaaggatccgacagtaatgaagctctaccctcggccaatattaccaaaggcgagaaggacgaagtcgtcgtggaggaaatcgacaagcctcaagaggatatcgacagtcagttcgaagcaactgtccgtcgtgccctggcacctttcaacgacgaggagaagcccgagcccaaggatctcgaggactcgtacaagtctttccgtacaatgctggttgttctctggctcttctccaactgtctcttggctgttgcgattactagcgataatttcgacgctttgacaaaggtaagctcaaatttgcttttgttccatttttcatatccagtgaagctaacatatcttgtttccagaaccaaaacacggccaggactgcctctttcttccagttcttgctgttctcgaccgctttcctctctttgatccgtttcatcggattcctctggttcttgggcaagaccggtataatgtgctgcattgcccgtcgctaagcgacagccgagtacgagagtcgcggcaacttcacgacttatgacaataagcatggtggtatttcatcatggtcgagcacgaagcttgcaaaagatcgtatcgcccatgtgtggattttcacacagaacacaccagccccattgaactactgatggccaggacagtcca

SEQ ID NO: 3 - protein, 929 aa, Magnaporthe grisea
MAYRGAGGPGGGRDYDGHNMQDLNPHSQYSNVQLPPGHEQEDEAHRSLLTQGTTLYDHDRLGAHTPPVRPVSAYSLTESYAPNAPTTVPGSAVGASPSPFQNDYGVSSGYQGAMGGHADDGFPIGGGDPQQGHPYDTEDSWVQRQNPNAAPQGGGLKRYATRKVKLVQGSVLSIDYNVPSAIRNAVQPKYREQEGTNEEFIKMRYTAATCDPNDFTLKNGYDLRPRMYNRHTELLIAITYYNEDKVLLSRTLHGVMQNIRDIVNLKKSTFWNKGGPAWQKIVVCLVFDGIEKTDKNVLDVLATIGIYQDGVVKKDVHGQETVAHIFEYTTQLSVTPSQQLIRPQDDGPNTLPPVQFIFCLKAKNSKKINSHRWLFNAFGRILNPEVCILLDAGTKPSSRSLLGLWEGFYNDKDLGGACGEIHAMLGKGGRKLLNPLVAVQNFEYKISNILDKPLESAFGYVSVLPGAFSAYRFRAIMGRPLEQYFHGDHTLSKILGKKGIEGMNIFKKNMFLAEDRILCFELVAKAGQKWHLSYIKAAKGETDVPEGAAEFISQRRRWLNGSFAATLYSLMHFGRMYKSGHNIIRMFFFHVQLIYNILNVIFTWFSLASYWLTTTVIMDLVGNPQVGQNAREGWPFGNTVTPLFNAVLKYIYLAFVILQFILALGNRPKGSKYTYVTSFFVFSVIQAYILVLSGYLVVQAFQTPIGEQIKTDTAKDFMDSIFGKSGAAGVILLALIAIYGIYFIASFMYLDPWHMFHSFPYYMLLMSTYINILMVYAFNNWHDVSWGTKGSDSNEALPSANITKGEKDEVVVEEIDKPQEDIDSQFEATVRRALAPFNDEEKPEPKDLEDSYKSFRTMLVVLWLFSNCLLAVAITSDNFDALTKNQNTARTASFFQFLLFSTAFLSLIRFIGFLWFLGKTGIMCCIARR

(19) United States
(12) Patent Application Publication
(10) Pub. No.: US 2003/0228645 A1
(43) Pub. Date: Dec. 11, 2003

(54) Methods for the identification of inhibitors of chitin synthase 2 as antibiotics

(76) Inventors: Sheryl Frank, Durham, NC (US); Blaise Darveaux, Hillsborough, NC (US); Sanjoy Mahanty, Chapel Hill, NC (US); Ryan Heiniger, Raleigh, NC (US); Amy Skalchunes, Raleigh, NC (US); Huaqin Pan, Apex, NC (US); Rex Tarpey, Apex, NC (US); Jeffrey Shuster, Chapel Hill, NC (US); Matthew M. Tanzer, Durham, NC (US); Lisbeth Hamer, Durham, NC (US); Kiichi Adachi, Tokyo (JP); M. DeZwaan Todd, Apex, NC (US); Sze-Chung Lo, Shun Lee Estate (HK); Maria Victoria Montenegro-Chamorro, Morrisville, NC (US)

Correspondence Address: PARADIGM GENETICS, INC, 108 ALEXANDER DRIVE, P O BOX 14528, RTP, NC, 27709-4528, US

(21) Appl. No.: 10/436,323

(22) Filed: May 12, 2003

Related U.S. Application Data
(60) Provisional application No. 60/381,159, filed on May 16, 2002.

(51) Int. Cl. C12Q 1/34; C12Q 1/18

(52) U.S. Cl. 435/18; 435/32

(57) ABSTRACT

The present inventors have discovered that Chitin Synthase 2 is essential for fungal pathogenicity. Specifically, the inhibition of Chitin Synthase 2 gene expression in fungi results in no signs of successful infection or lesions. Thus, Chitin Synthase 2 can be used as a target for the identification of antibiotics, preferably antifungals. Accordingly, the present invention provides methods for the identification of compounds that inhibit Chitin Synthase 2 expression or activity. The methods of the invention are useful for the identification of antibiotics, preferably antifungals.

### RELATED APPLICATIONS

This applications claims the benefit of U.S. Application No. 60/381,159 filed May 16, 2002, herein incorporated in its entirety by reference.

### FIELD OF THE INVENTION

The invention relates generally to methods for the identification of antibiotics, preferably antifungals that affect the biosynthesis of chitin.

### BACKGROUND OF THE INVENTION

Filamentous fungi are the causal agents responsible for many serious pathogenic infections of plants and animals. Since fungi are eukaryotes, and thus more similar to their host organisms than, for example bacteria, the treatment of infections by fungi poses special risks and challenges not encountered with other types of infections. One such fungus is Magnaporthe grisea, the fungus that causes rice blast disease. It is an organism that poses a significant threat to food supplies worldwide. Other examples of plant pathogens of economic importance are well known. Organisms classified as oomycetes include the genera Albugo, Aphanomyces, Bremia, Peronospora, Phytophthora, Plasmodiophora, Plasmopara, Pseudoperonospora, Pythium, Sclerophthora, and others. Oomycetes are significant plant pathogens and are sometimes classified along with the true fungi.

Human diseases caused by filamentous fungi include life-threatening lung and disseminated diseases, often resulting from infections by Aspergillus fumigatus. Other fungal diseases in animals are caused by fungi in the genera, Fusarium, Blastomyces, Microsporum, Trichophyton, Epidermophyton, Candida, Histoplamsa, Pneumocystis, Cryptococcus, other Aspergilli, and others. The control of fungal diseases in plants and animals is usually mediated by chemicals that inhibit the growth, proliferation, and/or pathogenicity of the fungal organisms. To date, there are less than twenty known modes-of-action for plant protection fungicides and human antifungal compounds.

A pathogenic organism has been defined as an organism that causes, or is capable of causing disease. Pathogenic organisms propagate on or in tissues and may obtain nutrients and other essential materials from their hosts. A substantial amount of work concerning filamentous fungal pathogens has been performed with the human pathogen, Aspergillus fumigatus. Shibuya et al. (Shibuya, K., M. Takaoka, et al. (1999) Microb Pathog 27: 123-31 (PMID: 10455003)) have shown that the deletion of either of two suspected pathogenicity related genes encoding an alkaline protease or a hydrophobin (rodlet) respectively, did not reduce mortality of mice infected with these mutant strains. Smith et al. (Smith, J. M., C. M. Tang, et al. (1994) Infect Immun 62: 5247-54 (PMID: 7960101)) showed similar results with alkaline protease and the ribotoxin restrictocin; Aspergillus fumigatus strains mutated for either of these genes were fully pathogenic to mice. Reichard et al. (Reichard, U., M. Monod, et al. (1997) J Med Vet Mycol 35: 189-96 (PMID: 9229335)) showed that deletion of the suspected pathogenicity gene encoding aspergillopepsin (PEP) in Aspergillus fumigatus had no effect on mortality in a guinea pig model system, and Aufauvre-Brown et al (Aufauvre-Brown, A., E. Mellado, et al. (1997) Fungal Genet Biol 21: 141-52 (PMID: 9073488)) showed no effects of a chitin synthase mutation on pathogenicity. However, not all experiments produced negative results. Ergosterol is an important membrane component found in fungal organisms. Pathogenic fungi that lack key enzymes in this biochemical pathway might be expected to be non-pathogenic since neither the plant nor animal hosts contain this particular sterol. Many antifungal compounds that affect this biochemical pathway have been previously described. (U.S. Pat. Nos. 4,920,109; 4,920,111; 4,920,112; 4,920,113; and 4,921,844; Fungicides in Crop Protection Cambridge, University Press (1990)). D'Enfert et al. (D'Enfert, C., M. Diaquin, et al. (1996) Infect Immun 64: 4401-5 (PMID: 8926121)) showed that an Aspergillus fumigatus strain mutated in an orotidine 5′-phosphate decarboxylase gene was entirely non-pathogenic in mice, and Brown et al. (Brown, J. S., A. Aufauvre-Brown, et al. (2000) Mol Microbiol 36:1371-80 (PMID: 10931287)) observed a non-pathogenic result when genes involved in the synthesis of para-aminobenzoic acid were mutated. Some specific target genes have been described as having utility for the screening of inhibitors of plant pathogenic fungi. U.S. Pat. No. 6,074,830, issued to Bacot et al. describes the use of 3,4-dihydroxy-2-butanone 4-phosphate synthase, and U.S. Pat. No. 5,976,848, issued to Davis et al. describes the use of dihydroorotate dehydrogenase for potential screening purposes.

There are also a number of papers that report less clear results, showing neither full pathogenicity nor non-pathogenicity of mutants. Hensel et al. (Hensel, M., H. N. Arst, Jr., et al. (1998) Mol Gen Genet 258: 553-7 (PMID: 9669338)) showed only moderate effects of the deletion of the area transcriptional activator on the pathogenicity of Aspergillus fumigatus.

Therefore, it is not currently possible to determine which specific growth materials may be readily obtained by a pathogen from its host, and which materials may not. We have found that Magnaporthe grisea that are deficient in a class III chitin synthase are non-pathogenic on their host organism. Nothing in the literature to date demonstrates an anti-pathogenic effect of the knock-out, over-expression, antisense expression, or inhibition of the genes or gene products involved in chitin biosynthesis in filamentous fungi. Thus, it has not been shown that the de novo biosynthesis of chitin is essential for fungal pathogenicity. Thus, it would be desirable to determine the utility of the enzymes involved in chitin biosynthesis for evaluating antibiotic compounds, especially fungicides. If a fungal biochemical pathway or specific gene product in that pathway is shown to be required for fungal pathogenicity, various formats of in vitro and in vivo screening assays may be put in place to discover classes of chemical compounds that react with the validated target gene, gene product, or biochemical pathway, and are thus candidates for antifungal, biocide, and biostatic materials.

### SUMMARY OF THE INVENTION

Surprisingly, the present inventors have discovered that in vivo disruption of the gene encoding Chitin Synthase 2 in Magnaporthe grisea prevents or inhibits the pathogenicity of the fungus. Thus, the present inventors have discovered that Chitin Synthase 2 is essential for normal rice blast pathogenicity, and can be used as a target for the identification of antibiotics, preferably fungicides. Accordingly, the present invention provides methods for the identification of compounds that inhibit Chitin Synthase 2 expression or activity. The methods of the invention are useful for the identification of antibiotics, preferably fungicides.

### BRIEF DESCRIPTION OF THE FIGURES

FIG. 1 shows the reaction performed by Chitin Synthase 2 (CHS2) reaction. The Substrates/Products are UDP-N-acetyl-D-glucosamine+[1,4-N-Acetyl-beta-D-glucosaminyl]n and the Products/Substrates are [1,4-N-Acetyl-beta-D-glucosaminyl]n+1 and UDP. The function of the Chitin Synthase 2 enzyme is the interconversion of UDP-N-acetyl-D-glucosamine+[1,4-N-Acetyl-beta-D-glucosaminyl]n to [1,4-N-Acetyl-beta-D-glucosaminyl]n+1 and UDP. This reaction is part of the chitin biosynthesis pathway.

FIG. 2 shows a digital image showing the effect of CHS2 gene disruption on Magnaporthe grisea pathogenicity using whole plant infection assays. Rice variety CO39 was inoculated with wild-type and the transposon insertion strains, KO1-5, KO1-17, and KO1-14. KO1-14 represents an ectopic transformant in which the transposon containing DNA fragment integrated at a nonhomologous site within the fungal genome and does not eliminate CHS2 activity. Leaf segments were imaged at five days post-inoculation.

### DETAILED DESCRIPTION OF THE INVENTION

Unless otherwise indicated, the following terms are intended to have the following meanings in interpreting the present invention.

The term “antibiotic” refers to any substance or compound that when contacted with a living cell, organism, virus, or other entity capable of replication, results in a reduction of growth, viability, or pathogenicity of that entity.

The term “binding” refers to a non-covalent or a covalent interaction, preferably non-covalent, that holds two molecules together. For example, two such molecules could be an enzyme and an inhibitor of that enzyme. Non-covalent interactions include hydrogen bonding, ionic interactions among charged groups, van der Waals interactions and hydrophobic interactions among nonpolar groups. One or more of these interactions can mediate the binding of two molecules to each other.

The term “biochemical pathway” or “pathway” refers to a connected series of biochemical reactions normally occurring in a cell, or more broadly a cellular event such as cellular division or DNA replication. Typically, the steps in such a biochemical pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene if the presence of the agent stops or substantially reduces the completion of the series of steps in that pathway. Such an agent, may, but does not necessarily, act directly on the expression product of that particular gene.

As used herein, “chain length” refers to the number of covalently linked constitutional repeating units per polymer. For example, in the case of a single polymer of chitin, chain length is indicated by the variable, n, in the formula [1,4-N-Acetyl-beta-D-glucosaminyl]n where the addition of one constitutional unit is indicated as n+1. For chitin, one constitutional unit is N-acetyl-D-glucosamine.

As used herein, the term “chitin” refers to [1,4-N-Acetyl-beta-D-glucosaminyl]n, when n>1, and is also known as poly-[1->4]-beta-N-acetyl-D-glucosamine. Chitins are polymers of N-acetyl-D-glucosamine. A polymer composed totally of N-acetyl-D-glucosamine is called chitin, and one composed totally of D-glucosamine is called chitosan. These polymers and those made up of a mixture of glucosamine and acetylglucosamine are known collectively as glucoaminoglycans. As used herein, “chitin” encompasses the definitions of“starter chitin,” and “shortened chitin” and “extended chitin,” which are used in part herein to distinguish the substrate of the Chitin Synthase 2 reaction from the products. Chitin Synthase 2 catalyses the interconversion of UDP-N-acetyl-D-glucosamine and [1,4-N-Acetyl-beta-D-glucosaminyl]n with [1,4-N-Acetyl-beta-D-glucosaminyl]n+1 and UDP.

As used herein, the terms “Chitin Synthase 2 (CHS2)”, Chitin synthase, Chitin-UDP N-acetylglucosaminyltransferase, UDP-N-acetyl-D-glucosamine:chitin 4-beta-N-acetylglucosaminyl-transferase, and and “Chitin Synthase 2 (CHS2) polypeptide” refer to an enzyme that catalyzes the reversible interconversion of UDP-N-acetyl-D-glucosamine+[1,4-N-Acetyl-beta-D-glucosaminyl]n with [1,4-N-Acetyl-beta-D-glucosaminyl]n+1 and UDP. Although the protein and/or the name of the gene that encodes the protein may differ between species, the terms “CHS2” and “CHS2 gene product” are intended to encompass any polypeptide that catalyzes the reversible interconversion of UDP-N-acetyl-D-glucosamine+[1,4-N-Acetyl-beta-D-glucosaminyl]n with [1,4-N-Acetyl-beta-D-glucosaminyl]n+1 and UDP.

As used herein, the term “CoA” means coenzyme A.

As used herein, the term “conditional lethal” refers to a mutation permitting growth and/or survival only under special growth or environmental conditions.

As used herein, the term “cosmid” refers to a hybrid vector, used in gene cloning, that includes a cos site (from the lambda bacteriophage). It also contains drug resistance marker genes and other plasmid genes. Cosmids are especially suitable for cloning large genes or multigene fragments.

As used herein, the term “dominant allele” refers to a dominant mutant allele in which a discernable mutant phenotype can be detected when this mutation is present in an organism that also contains a wild type (non-mutant), recessive allele, or other dominant allele.

As used herein, the term “ELISA” means enzyme-linked immunosorbent assay.

As used herein, the term “extended chitin” refers to a chitin polymer or population of chitin that increases in total amount and/or chain length. Changes in length are often measured by incorporation or release of labeled polymer subunits, or changes in polymer weight.

“Fungi” (singular: fungus) refers to whole fungi, fungal organs and tissues (e.g., asci, hyphae, pseudohyphae, rhizoid, sclerotia, sterigmata, spores, sporodochia, sporangia, synnemata, conidia, ascostroma, cleistothecia, mycelia, perithecia, basidia and the like), spores, fungal cells and the progeny thereof. Fungi are a group of organisms (about 50,000 known species), including, but not limited to, mushrooms, mildews, moulds, yeasts, etc., comprising the kingdom Fungi. They can either exist as single cells or make up a multicellular body called a mycelium, which consists of filaments known as hyphae. Most fungal cells are multinucleate and have cell walls, composed chiefly of chitin. Fungi exist primarily in damp situations on land and, because of the absence of chlorophyll and thus the inability to manufacture their own food by photosynthesis, are either parasites on other organisms or saprotrophs feeding on dead organic matter. The principal criteria used in classification are the nature of the spores produced and the presence or absence of cross walls within the hyphae. Fungi are distributed worldwide in terrestrial, freshwater, and marine habitats. Some live in the soil. Many pathogenic fungi cause disease in animals and man or in plants, while some saprotrophs are destructive to timber, textiles, and other materials. Some fungi form associations with other organisms, most notably with algae to form lichens.

As used herein, the term “fungicide,” “antifungal,” or “antimycotic” refers to an antibiotic substance or compound that kills or suppresses the growth, viability, or pathogenicity of at least one fungus, fungal cell, fungal tissue or spore.

As used in this disclosure, the terms “growth” or “cell growth” of an organism refers to an increase in mass, density, or number of cells of said organism. Some common methods for the measurement of growth include the determination of the optical density of a cell suspension, the counting of the number of cells in a fixed volume, the counting of the number of cells by measurement of cell division, the measurement of cellular mass or cellular volume, and the like.

As used in this disclosure, the term “growth conditional phenotype” indicates that a fungal strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a fungal strain having a heat-sensitive phenotype) exhibits significantly different growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

As used herein, the term “heterologous CHS2” means either a nucleic acid encoding a polypeptide or a polypeptide, wherein the polypeptide has at least 50%, 55%, 60%, 65%, 70%, 75%, 80%, 85%, 90%, 91%, 92%, 93%, 94%, 95%, 96%, 97%, 98%, or 99% sequence identity or each integer unit of sequence identity from 50-100% in ascending order to M. grisea CHS2 protein (SEQ ID NO: 3) and at least 10%, 25%, 50%, 75%, 80%, 90%, 95%, or 99% activity or each integer unit of activity from 10-100% in ascending order of the activity of M. grisea CHS2 protein (SEQ ID NO: 3). An example of a heterologous CHS2 includes, but is not limited to, CHS3 from Neurospora crassa (GENBANK: 83753).

As used herein, the term “His-Tag” refers to an encoded polypeptide consisting of multiple consecutive histidine amino acids.

As used herein, the terms “hph”, “hygromycin B phosphotransferase”, and “hygromycin resistance gene” refer to the E. coli hygromycin phosphotransferase gene or gene product.

As used herein, the term “imperfect state” refers to a classification of a fungal organism having no demonstrable sexual life stage.

The term “inhibitor,” as used herein, refers to a chemical substance that inactivates the enzymatic activity of Chitin Synthase 2 or substantially reduces the level of enzymatic activity, wherein “substantially” means a reduction at least as great as the standard deviation for a measurement, preferably a reduction by 50%, more preferably a reduction of at least one magnitude, i.e. to 10%. The inhibitor may function by interacting directly with the enzyme, a cofactor of the enzyme, the substrate of the enzyme, or any combination thereof.

A polynucleotide may be “introduced” into a fungal cell by any means known to those of skill in the art, including transfection, transformation or transduction, transposable element, electroporation, particle bombardment, infection and the like. The introduced polynucleotide may be maintained in the cell stably if it is incorporated into a non-chromosomal autonomous replicon or integrated into the fungal chromosome. Alternatively, the introduced polynucleotide may be present on an extra-chromosomal non-replicating vector and be transiently expressed or transiently active.

As used herein, the term “knockout” or “gene disruption” refers to the creation of organisms carrying a null mutation (a mutation in which there is no active gene product), a partial null mutation or mutations, or an alteration or alterations in gene regulation by interrupting a DNA sequence through insertion of a foreign piece of DNA. Usually the foreign DNA encodes a selectable marker.

The term “method of screening” means that the method is suitable, and is typically used, for testing for a particular property or effect in a large number of compounds. Typically, more than one compound is tested simultaneously (as in a 96-well microtiter plate), and preferably significant portions of the procedure can be automated. “Method of screening” also refers to the determination of a set of different properties or effects of one compound simultaneously.

As used herein, the term “mutant form” of a gene refers to a gene which has been altered, either naturally or artificially, changing the base sequence of the gene. The change in the base sequence may be of several different types, including changes of one or more bases for different bases, deletions, and/or insertions, such as by a transposon. By contrast, a normal form of a gene (wild type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “Ni-NTA” refers to nickel sepharose.

As used herein, a “normal” form of a gene (wild type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “one form” of a gene is synonymous with the term “gene”, and a “different form” of a gene refers to a gene that has greater than 49% sequence identity and less than 100% sequence identity with said first form.

As used herein, the term “pathogenicity” refers to a capability of causing disease.

The “percent (%) sequence identity” between two polynucleotide or two polypeptide sequences is determined according to the either the BLAST program (Basic Local Alignment Search Tool; (Altschul, S. F., W. Gish, et al. (1990) J Mol Biol 215: 403-10 (PMID: 2231712)) at the National Center for Biotechnology or using Smith Waterman Alignment (Smith, T. F. and M. S. Waterman (1981) J Mol Biol 147: 195-7 (PMID: 7265238)) as incorporated into GENEMATCHER PLUS. It is understood that for the purposes of determining sequence identity when comparing a DNA sequence to an RNA sequence, a thymine nucleotide is equivalent to a uracil nucleotide.

By “polypeptide” is meant a chain of at least two amino acids joined by peptide bonds. The chain may be linear, branched, circular or combinations thereof. Preferably, polypeptides are from about 10 to about 1000 amino acids in length, more preferably 10-50 amino acids in length. The polypeptides may contain amino acid analogs and other modifications, including, but not limited to glycosylated or phosphorylated residues.

As used herein, the term “proliferation” is synonymous to the term “growth.”

As used herein, “semi-permissive conditions” are conditions in which the relevant culture parameter for a particular growth conditional phenotype is intermediate between permissive conditions and non-permissive conditions. Consequently, in semi-permissive conditions an organism having a growth conditional phenotype will exhibit growth rates intermediate between those shown in permissive conditions and non-permissive conditions. In general, such intermediate growth rate may be due to a mutant cellular component which is partially functional under semi-permissive conditions, essentially fully functional under permissive conditions, and is non-functional or has very low function under non-permissive conditions, where the level of function of that component is related to the growth rate of the organism. An intermediate growth rate may also be a result of a nutrient substance or substances that are present in amounts not sufficient for optimal growth rates to be achieved.

“Sensitivity phenotype” refers to a phenotype that exhibits either hypersensitivity or hyposensitivity.

As used herein, the term “shortened chitin” refers to a chitin polymer or population of chitin that decreases in total amount and/or chain length. Changes in length are often measured by incorporation or release of labeled polymer subunits, or changes in polymer weight.

The term “specific binding” refers to an interaction between Chitin Synthase 2 and a molecule or compound, wherein the interaction is dependent upon the primary amino acid sequence and/or the conformation of Chitin Synthase 2.

As used herein, the term “starter chitin” refers to the chitin present and the state of its composition at the beginning of a reaction or a period over which changes in the amount of chitin present, and/or its state of composition, such as the length/number of N-acetyl-D-glucosamine groups for a particular chitin polymer, or population or sub-population of chitin polymers, are measured. The term encompasses the variety of populations of chitin that might be present at the start of such a reaction or measurement period. Such populations might include, but are not limited to, isolated chitin of uniform chain length, isolated chitin of mixed chain length, unpurified chitin of mixed chain length as might be found in a cell lysate, etc. “Starter chitin” can also be used to refer to a chitin polymer or population of chitin polymers unchanged, or meeting the criteria for “unchanged”, after a reaction or period of measurement. A chitin polymer or population of chitin that decreases in total amount and/or chain length is referred to as “shortened chitin”. A chitin polymer or population of chitin that increases in total amount and/or chain length is referred to as “extended chitin”. Changes in length are often measured by incorporation or release of labeled polymer subunits, or changes in polymer weight.

“Transform,” as used herein, refers to the introduction of a polynucleotide (single or double stranded DNA, RNA, or a combination thereof) into a living cell by any means. Transformation may be accomplished by a variety of methods, including, but not limited to, electroporation, polyethylene glycol mediated uptake, particle bombardment, agrotransformation, and the like. This process may result in transient or stable expression of the transformed polynucleotide. By “stably transformed” is meant that the sequence of interest is integrated into a replicon in the cell, such as a chromosome or episome. Transformed cells encompass not only the end product of a transformation process, but also the progeny thereof which retain the polynucleotide of interest.

For the purposes of the invention, “transgenic” refers to any cell, spore, tissue or part, that contains all or part of at least one recombinant polynucleotide. In many cases, all or part of the recombinant polynucleotide is stably integrated into a chromosome or stable extra-chromosomal element, so that it is passed on to successive generations.

As used herein, the term “transposition” refers to a complex genetic rearrangement process involving the movement or copying of a polynucleotide (transposon) from one location and insertion into another, often within or between a genome or genomes, or DNA constructs such as plasmids, bacmids, and cosmids.

The term “transposon” as used herein is interchangeable with the following terms: “transposable element,” “transposable genetic element,” “mobile element,” or “jumping gene,” all of which refer generally to a mobile DNA element. Transposons can disrupt gene expression or cause deletions and inversions, and hence affect both the genotype and phenotype of the organisms concerned. The mobility of transposable elements has long been used in genetic manipulation, to introduce genes or other information into the genome of certain model systems.

As used herein, the term “TWEEN 20” means sorbitan mono-9-octadecenoate poly(oxy-1,1-ethanediyl).

As used herein, the term “UDP” means uridine diphosphate.

As used in this disclosure, the term “viability” of an organism refers to the ability of an organism to demonstrate growth under conditions appropriate for said organism, or to demonstrate an active cellular function. Some examples of active cellular functions include respiration as measured by gas evolution, secretion of proteins and/or other compounds, dye exclusion, mobility, dye oxidation, dye reduction, pigment production, changes in medium acidity, and the like.

The present inventors have discovered that disruption of the CHS2 gene and/or gene product inhibits the pathogenicity of Magnaporthe grisea. Thus, the inventors are the first to demonstrate that Chitin Synthase 2 is a target for antibiotics, preferably antifungals.

Examples of plant pathogens of economic importance include the pathogens in the genera Agaricus, Alternaria, Anisogramma, Anthracoidea, Antrodia, Apiognomonia, Apiosporina, Armillaria, Ascochyta, Aspergillus, Bipolaris, Bjerkandera, Botryosphaeria, Botrytis, Ceratobasidium, Ceratocystis, Cercospora, Cercosporidium, Cerotelium, Cerrena, Chondrostereum, Chryphonectria, Chrysomyxa, Cladosporium, Claviceps, Cochliobolus, Coleosporium, Colletotrichium, Colletotrichum, Corticium, Corynespora, Cronartium, Cryphonectria, Cryptosphaeria, Cyathus, Cymadothea, Cytospora, Daedaleopsis, Diaporthe, Didymella, Diplocarpon, Diplodia, Discohainesia, Discula, Dothistroma, Drechslera, Echinodontium, Elsinoe, Endocronartium, Endothia, Entyloma, Epichloe, Erysiphe, Exobasidium, Exserohilum, Fomes, Fomitopsis, Fusarium, Gaeumannomyces, Ganoderma, Gibberella, Gloeocercospora, Gloeophyllum, Gloeoporus, Glomerella, Gnomoniella, Guignardia, Gymnosporangium, Helminthosporium, Herpotrichia, Heterobasidion, Hirschioporus, Hypodermella, Inonotus, Irpex, Kabatiella, Kabatina, Laetiporus, Laetisaria, Lasiodiplodia, Laxitextum, Leptographium, Leptosphaeria, Leptosphaerulina, Leucytospora, Linospora, Lophodermella, Lophodermium, Macrophomina, Magnaporthe, Marssonina, Melampsora, Melampsorella, Meria, Microdochium, Microsphaera, Monilinia, Monochaetia, Morchella, Mycosphaerella, Myrothecium, Nectria, Nigrospora, Ophiosphaerella, Ophiostoma, Penicillium, Perenniporia, Peridermium, Pestalotia, Phaeocryptopus, Phaeolus, Phakopsora, Phellinus, Phialophora, Phoma, Phomopsis, Phragmidium, Phyllachora, Phyllactinia, Phyllosticta, Phymatotrichopsis, Pleospora, Podosphaera, Pseudopeziza, Pseudoseptoria, Puccinia, Pucciniastrum, Pyricularia, Rhabdocline, Rhizoctonia, Rhizopus, Rhizosphaera, Rhynchosporium, Rhytisma, Schizophyllum, Schizopora, Scirrhia, Sclerotinia, Sclerotium, Scytinostroma, Septoria, Setosphaera, Sirococcus, Spaerotheca, Sphaeropsis, Sphaerotheca, Sporisorium, Stagonospora, Stemphylium, Stenocarpella, Stereum, Taphrina, Thielaviopsis, Tilletia, Trametes, Tranzschelia, Trichoderma, Tubakia, Typhula, Uncinula, Urocystis, Uromyces, Ustilago, Valsa, Venturia, Verticillium, Xylaria, and others. Related organisms in the classification, oomycetes, include the genera Albugo, Aphanomyces, Bremia, Peronospora, Phytophthora, Plasmodiophora, Plasmopara, Pseudoperonospora, Pythium, Sclerophthora, and others, are known significant plant pathogens and can be classified along with the true fungi.

Human diseases that are caused by filamentous fungi include life-threatening lung and disseminated diseases, often a resulting from infections by Aspergillus fumigatus. Other fungal diseases in animals are caused by fungi in the genera, Fusarium, Blastomyces, Microsporum, Trichophyton, Epidermophyton, Candida, Histoplamsa, Pneumocystis, Cryptococcus, other Aspergilli, and others. The control of fungal diseases in plants and animals is usually mediated by chemicals that inhibit the growth, proliferation, and/or pathogenicity of the fungal organisms.

The present invention provides methods for identifying compounds that inhibit CHS2 gene expression or biological activity of its gene product(s). Such methods include ligand binding assays, assays for enzyme activity, cell-based assays, and assays for CHS2 gene expression. Any compound that is a ligand for Chitin Synthase 2 may have antibiotic activity. For the purposes of the invention, “ligand” refers to a molecule that will bind to a site on a polypeptide. The compounds identified by the methods of the invention are useful as antibiotics.

Thus, in one embodiment, the invention provides a method for identifying a test compound as a candidate for an antibiotic, comprising: contacting a Chitin Synthase 2 polypeptide with a test compound and detecting the presence or absence of binding between the test compound and the Chitin Synthase 2 polypeptide, such that binding indicates that the test compound is a candidate for an antibiotic.

The Chitin Synthase 2 protein may have the amino acid sequence of a naturally occurring Chitin Synthase 2 found in a fungus, animal, plant, or microorganism, or may have an amino acid sequence derived from a naturally occurring sequence. Preferably the Chitin Synthase 2 is a fungal Chitin Synthase 2. The cDNA (SEQ ID NO: 1) encoding the Chitin Synthase 2 protein, the genomic DNA (SEQ ID NO: 2) encoding the M. grisea protein, and the polypeptide (SEQ ID NO: 3) can be found herein.

In one aspect, the invention also provides for a polypeptide consisting essentially of SEQ ID NO: 3. For the purposes of the invention, a polypeptide consisting essentially of SEQ ID NO: 3 has at least 85% sequence identity with SEQ ID NO: 3 and catalyses the interconversion of UDP-N-acetyl-D-glucosamine+[1,4-N-Acetyl-beta-D-glucosaminyl]n with [1,4-N-Acetyl-beta-D-glucosaminyl]n+1 and UDP with at least 10% of the activity of SEQ ID NO: 3. Preferably, the polypeptide consisting essentially of SEQ ID NO: 3 has at least 90% sequence identity with SEQ ID NO: 3, more preferably the sequence identity is at least 95% or 97 or 99%, or any integer from 80-100% sequence identity in ascending order. And, preferably, the polypeptide consisting essentially of SEQ ID NO: 3 has at least 25%, at least 50%, at least 75% or at least 90% of the activity of M. grisea Chitin Synthase 2, or any integer from 60-100% activity in ascending order.

By “fungal Chitin Synthase 2” is meant an enzyme that can be found in at least one fungus, and which catalyzes the interconversion of UDP-N-acetyl-D-glucosamine+[1,4-N-Acetyl-beta-D-glucosaminyl]n with [1,4-N-Acetyl-beta-D-glucosaminyl]n+1 and UDP. The Chitin Synthase 2 may be from any of the fungi, including ascomycota, zygomycota, basidiomycota, chytridiomycota, and lichens.

In one embodiment, the Chitin Synthase 2 is a Magnaporthe Chitin Synthase 2. Magnaporthe species include, but are not limited to, Magnaporthe rhizophila, Magnaporthe salvinii, Magnaporthe grisea and Magnaporthe poae and the imperfect states of Magnaporthe in the genus Pyricularia. Preferably, the Magnaporthe Chitin Synthase 2 is from Magnaporthe grisea.

In various embodiments, the Chitin Synthase 2 can be from Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), and the like.

Fragments of a Chitin Synthase 2 polypeptide may be used in the methods of the invention, preferably if the fragments include an intact or nearly intact epitope that occurs on the biologically active wildtype Chitin Synthase 2. The fragments comprise at least 10 consecutive amino acids of a Chitin Synthase 2. Preferably, the fragment comprises at least 15, 20, 25, 30, 35, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, or at least 920 consecutive amino acids residues of a Chitin Synthase 2. In one embodiment, the fragment is from a Magnaporthe Chitin Synthase 2. Preferably, the fragment contains an amino acid sequence conserved among fungal Chitin Synthase 2s.

Polypeptides having at least 50% sequence identity with a fungal Chitin Synthase 2 are also useful in the methods of the invention. Preferably, the sequence identity is at least 60%, more preferably the sequence identity is at least 70%, most preferably the sequence identity is at least 80% or 90 or 95 or 99%, or any integer from 60-100% sequence identity in ascending order.

In addition, it is preferred that the polypeptide has at least 10% of the activity of a fungal Chitin Synthase 2. More preferably, the polypeptide has at least 25%, at least 50%, at least 75% or at least 90% of the activity of a fungal Chitin Synthase 2. Most preferably, the polypeptide has at least 10%, at least 25%, at least 50%, at least 75% or at least 90% of the activity of the M. grisea Chitin Synthase 2 protein.

Thus, in another embodiment, the invention provides a method for identifying a test compound as a candidate for a fungicide, comprising: contacting a test compound with at least one polypeptide selected from the group consisting of: a polypeptide having at least ten consecutive amino acids of a fungal Chitin Synthase 2; a polypeptide having at least 50% sequence identity with a fungal Chitin Synthase 2; and a polypeptide having at least 10% of the activity of a fungal Chitin Synthase 2; and detecting the presence and/or absence of binding between the test compound and the polypeptide, such that binding indicates that the test compound is a candidate for an antibiotic.

Any technique for detecting the binding of a ligand to its target may be used in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a Chitin Synthase 2 protein or a fragment or variant thereof, the unbound protein is removed and the bound Chitin Synthase 2 is detected. In a preferred embodiment, bound Chitin Synthase 2 is detected using a labeled binding partner, such as a labeled antibody. In a variation of this assay, Chitin Synthase 2 is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for an antibiotic, it can be tested for the ability to inhibit Chitin Synthase 2 enzymatic activity. The compounds can be tested using either in vitro or cell based assays. Alternatively, a compound can be tested by applying it directly to a fungus or fungal cell, or expressing it therein, and monitoring the fungus or fungal cell for changes or decreases in growth, development, viability, pathogenicity, or alterations in gene expression. Thus, in one embodiment, the invention provides a method for determining whether a compound identified as an antibiotic candidate by an above method has antifungal activity, further comprising: contacting a fungus or fungal cells with said antifungal candidate and detecting a decrease in the growth, viability, or pathogenicity of said fungus or fungal cells.

By decrease in growth, is meant that the antifungal candidate causes at least a 10% decrease in the growth of the fungus or fungal cells, as compared to the growth of the fungus or fungal cells in the absence of the antifungal candidate. By a decrease in viability is meant that at least 20% of the fungal cells, or portion of the fungus contacted with the antifungal candidate are nonviable. Preferably, the growth or viability will be decreased by at least 40%. More preferably, the growth or viability will be decreased by at least 50%, 75% or at least 90% or more. Methods for measuring fungal growth and cell viability are known to those skilled in the art. By decrease in pathogenicity, is meant that the antifungal candidate causes at least a 10% decrease in the disease caused by contact of the fungal pathogen with its host, as compared to the disease caused in the absence of the antifungal candidate. Preferably, the disease will be decreased by at least 40%. More preferably, the disease will be decreased by at least 50%, 75% or at least 90% or more. Methods for measuring fungal disease are well known to those skilled in the art, and include such metrics as lesion formation, lesion size, sporulation, respiratory failure, and/or death.

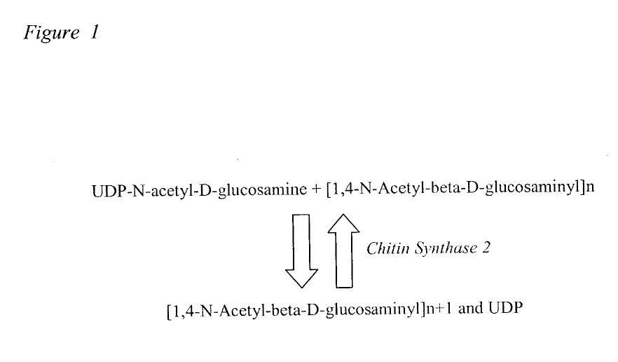

The ability of a compound to inhibit Chitin Synthase 2 activity can be detected using in vitro enzymatic assays in which the disappearance of a substrate or the appearance of a product is directly or indirectly detected. Chitin Synthase 2 catalyzes the irreversible or reversible reaction UDP-N-acetyl-D-glucosamine+[1,4-N-Acetyl-beta-D-glucosaminyl]n=[1,4-N-Acetyl-beta-D-glucosaminyl]n+1 and UDP (see FIG. 1) Methods for detection of UDP-N-acetyl-D-glucosamine, starter chitin, extended chitin, shortened chitin, and/or UDP, include spectrophotometry, mass spectroscopy, thin layer chromatography (TLC) and reverse phase HPLC.

Thus, the invention provides a method for identifying a test compound as a candidate for an antibiotic comprising: contacting UDP-N-acetyl-D-glucosamine and starter chitin with a Chitin Synthase 2; contacting UDP-N-acetyl-D-glucosamine and starter chitin with Chitin Synthase 2 and a test compound; and determining the change in concentration for at least one of the following: UDP-N-acetyl-D-glucosamine, starter chitin, extended chitin, and/or UDP, such that a change in concentration for any of the above substances indicates that the test compound is a candidate for an antibiotic.

An alternate method is provided by the invention for identifying a test compound as a candidate for an antibiotic, comprising: contacting starter chitin and UDP with a Chitin Synthase 2; contacting starter chitin and UDP with a Chitin Synthase 2 and a test compound; and determining the change in concentration for at least one of the following: UDP-N-acetyl-D-glucosamine, starter chitin, shortened chitin, and/or UDP, such that a change in concentration for any of the above substances indicates that the test compound is a candidate for an antibiotic.

Enzymatically active fragments of a fungal Chitin Synthase 2 are also useful in the methods of the invention. For example, an enzymatically active polypeptide comprising at least 100 consecutive amino acid residues of a fungal Chitin Synthase 2 may be used in the methods of the invention. In addition, an enzymatically active polypeptide having at least 50%, 60%, 70%, 80%, 90%, 95% or at least 98% sequence identity with a fungal Chitin Synthase 2 may be used in the methods of the invention. Most preferably, the polypeptide has at least 50% sequence identity with a fungal Chitin Synthase 2 and at least 10%, 25%, 75% or at least 90% of the activity thereof.

Thus, the invention provides a method for identifying a test compound as a candidate for an antibiotic comprising: contacting UDP-N-acetyl-D-glucosamine and starter chitin with a polypeptide selected from the group consisting of: a polypeptide having at least 50% sequence identity with a Chitin Synthase 2; a polypeptide having at least 50% sequence identity with a Chitin Synthase 2 and having at least 10% of the activity thereof; and a polypeptide comprising at least 100 consecutive amino acids of a Chitin Synthase 2; contacting UDP-N-acetyl-D-glucosamine and starter chitin with the polypeptide and a test compound; and determining the change in concentration for at least one of the following: UDP-N-acetyl-D-glucosamine, starter chitin, extended chitin, and/or UDP, such that a change in concentration for any of the above substances indicates that the test compound is a candidate for an antibiotic. An alterante method is provided by the invention for identifying a test compound as a candidate for an antibiotic comprising: contacting starter chitin and UDP with a polypeptide selected from the group consisting of: a polypeptide having at least 50% sequence identity with a Chitin Synthase 2; a polypeptide having at least 50% sequence identity with a Chitin Synthase 2 and at least 10% of the activity thereof; and a polypeptide comprising at least 100 consecutive amino acids of a Chitin Synthase 2; contacting starter chitin and UDP, with said polypeptide and a test compound; and determining the change in concentration for at least one of the following, UDP-N-acetyl-D-glucosamine, starter chitin, shortened chitin, and/or UDP, such that a change in concentration for any of the above substances indicates that the test compound is a candidate for an antibiotic.

For the in vitro enzymatic assays, Chitin Synthase 2 protein and derivatives thereof may be isolated from a fungus or may be recombinantly produced in and isolated from an archael, bacterial, fungal, or other eukaryotic cell culture. Preferably these proteins are produced using an E. coli, yeast, or filamentous fungal expression system. Methods for the purification of Chitin Synthase 2 may be described in Cabib et al. (1987) Methods Enzymol 138: 643-9 (PMID: 2955198). Other methods for the purification of Chitin Synthase 2 proteins and polypeptides are known to those skilled in the art.

As an alternative to in vitro assays, the invention also provides cell based assays. In one embodiment, the invention provides a method for identifying a test compound as a candidate for an antibiotic, comprising: measuring the expression of a Chitin Synthase 2 in a cell, cells, tissue, or an organism in the absence of a test compound; contacting the cell, cells, tissue, or organism with the test compound and measuring the expression of said Chitin Synthase 2 in the cell, cells, tissue, or organism; and comparing the expression of Chitin Synthase 2 such that a lower expression in the presence of the test compound indicates that the compound is a candidate for an antibiotic.

Expression of Chitin Synthase 2 can be measured by detecting the CHS2 primary transcript or mRNA, Chitin Synthase 2 polypeptide, or Chitin Synthase 2 enzymatic activity. Methods for detecting the expression of RNA and proteins are known to those skilled in the art. See, for example, Current Protocols in Molecular Biology Ausubel et al., eds., Greene Publishing and Wiley-Interscience, New York, 1995. The method of detection is not critical to the invention. Methods for detecting CHS2 RNA include, but are not limited to amplification assays such as quantitative reverse transcriptase-PCR, and/or hybridization assays such as Northern analysis, dot blots, slot blots, in-situ hybridization, transcriptional fusions using a CHS2 promoter fused to a reporter gene, DNA assays, and microarray assays.

Methods for detecting protein expression include, but are not limited to, immunodetection methods such as Western blots, ELISA assays, polyacrylamide gel electrophoresis, mass spectroscopy, and enzymatic assays. Also, any reporter gene system may be used to detect CHS2 protein expression. For detection using gene reporter systems, a polynucleotide encoding a reporter protein is fused in frame with CHS2, so as to produce a chimeric polypeptide. Methods for using reporter systems are known to those skilled in the art.

Chemicals, compounds or compositions identified by the above methods as modulators, preferably inhibitors, of CHS2 expression or activity can then be used to control fungal growth. Diseases such as rusts, mildews, and blights spread rapidly once established. Fungicides are thus routinely applied to growing and stored crops as a preventive measure, generally as foliar sprays or seed dressings. For example, compounds that inhibit fungal growth can be applied to a fungus or expressed in a fungus, in order to prevent fungal growth. Thus, the invention provides a method for inhibiting fungal growth, comprising contacting a fungus with a compound identified by the methods of the invention as having antifungal activity.

Antifungals and antifungal inhibitor candidates identified by the methods of the invention can be used to control the growth of undesired fungi, including ascomycota, zygomycota, basidiomycota, chytridiomycota, and lichens.

Examples of undesired fungi include, but are not limited to Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), diseases of animals such as infections of lungs, blood, brain, skin, scalp, nails or other tissues (Aspergillus fumigatus Aspergillus sp. Fusraium sp., Trichophyton sp., Epidermophyton sp., and Microsporum sp., and the like).

It is recognized in the art that determination of the growth of said organism in the paired media in the absence of any test compounds may be performed to control for any inherent differences in growth as a result of the different media. Growth and/or proliferation of an organism is measured by methods well known in the art such as optical density measurements, and the like. In a preferred embodiment, the organism is Magnaporthe grisea.

### EXPERIMENTAL

### Example 1

Construction of Plasmids with a Transposon Containing a Selectable Marker.

Construction of Sif transposon: Sif was constructed using the GPS3 vector from the GPS-M mutagenesis system from New England Biolabs, Inc. (Beverly, Mass.) as a backbone. This system is based on the bacterial transposon Tn7. The following manipulations were done to GPS3 according to Sambrook et al. (1989) Molecular Cloning, a Laboratory Manual, Cold Spring Harbor Laboratory Press. The kanamycin resistance gene (npt) contained between the Tn7 arms was removed by EcoRV digestion. The bacterial hygromycin B phosphotransferase (hph) gene (Gritz and Davies (1983) Gene 25: 179-88 (PMID: 6319235)) under control of the Aspergillus nidulans trpC promoter and terminator (Mullaney et al. (1985) Mol Gen Genet 199: 37-45 (PMID: 3158796)) was cloned by a HpaI/EcoRV blunt ligation into the Tn7 arms of the GPS3 vector yielding pSifl. Excision of the ampicillin resistance gene (bla) from pSifl was achieved by cutting pSifl with XmnI and BglI followed by a T4 DNA polymerase treatment to remove the 3′ overhangs left by the BglI digestion and religation of the plasmid to yield pSif. Top 10F′ electrocompetent E. coli cells (Invitrogen) were transformed with ligation mixture according to manufacturer's recommendations. Transformants containing the Sif transposon were selected on LB agar (Sambrook et al. (1989) Molecular Cloning, a Laboratory Manual) containing 50 ug/ml of hygromycin B (Sigma Chem. Co., St. Louis, Mo.).

### Example 2

Construction of a Fungal Cosmid Library

Cosmid libraries were constructed in the pcosKA5 vector (Hamer et al. (2001) Proc Natl Acad Sci USA 98: 5110-15 (PMID: 11296265)) as described in Sambrook et al. (1989) Molecular Cloning, a Laboratory Manual. Cosmid libraries were quality checked by pulsed-field gel electrophoresis, restriction digestion analysis, and PCR identification of single genes.

### Example 3

Construction of Cosmids with Transposon Insertion into Fungal Genes

Sif Transposition into a Cosmid: Transposition of Sif into the cosmid framework was carried out as described by the GPS-M mutagenesis system (New England Biolabs, Inc.). Briefly, 2 ul of the 10× GPS buffer, 70 ng of supercoiled pSIF, 8-12 ug of target cosmid DNA were mixed and taken to a final volume of 20 ul with water. 1 ul of transposase (TnsABC) was added to the reaction and incubated for 10 minutes at 37° C. to allow the assembly reaction to happen. After the assembly reaction, 1 ul of start solution was added to the tube, mixed well and incubated for 1 hour at 37° C. followed by heat inactivation of the proteins at 75° C. for 10 min. Destruction of the remaining untransposed pSif was done by PISceI digestion at 37° C. for 2 hours followed by 10 min incubation at 75° C. to inactivate the proteins. Transformation of Top 10F′ electro-competent cells (Invitrogen) was done according to manufacturers recommendations. Sif-containing cosmid transformants were selected by growth on LB agar plates containing 50 ug/ml of hygromycin B (Sigma Chem. Co.) and 100 ug/ml of Ampicillin (Sigma Chem. Co.).

### Example 4

High Throughput Preparation and Verification of Transposon Insertion into the M. grisea CHS2 Gene

E. coli strains containing cosmids with transposon insertions were picked to 96 well growth blocks (Beckman Co.) containing 1.5 ml of TB (Terrific Broth, Sambrook et al. (1989) Molecular Cloning, a Laboratory Manual, Cold Spring Harbor Laboratory Press) supplemented with 50 ug/ml of ampicillin. Blocks were incubated with shaking at 37° C. overnight. E. coli cells were pelleted by centrifugation and cosmids were isolated by a modified alkaline lysis method (Marra et al. (1997) Genome Res 7: 1072-84 (PMID: 9371743)). DNA quality was checked by electrophoresis on agarose gels. Cosmids were sequenced using primers from the ends of each transposon and commercial dideoxy sequencing kits (Big Dye Terminators, Perkin Elmer Co.). Sequencing reactions were analyzed on an ABI377 DNA sequencer (Perkin Elmer Co.).

DNA sequences adjacent to the site of the insertion were collected and used to search DNA and protein databases using the BLAST algorithms (Altschul et al. (1997) Nucleic Acids Res 25: 3389-3402 (PMID: 9254694)). A single insertion of SIF into the Magnaporthe grisea CHS2 gene was chosen for further analysis. This construct was designated cpgmra0011005c02 and it contains the SIF transposon approximately between amino acids 427 and 428 relative to the Neurospora crassa homologue, CHS3 (total length: 960 amino acids, GENBANK: 83753).

### Example 5

Preparation of CHS2 Cosmid DNA and Transformation of Magnaporthe grisea

Cosmid DNA from the CHS2 transposon tagged cosmid clone was prepared using QIAGEN Plasmid Maxi Kit (QIAGEN), and digested by PI-PspI (New England Biolabs, Inc.). Fungal electro-transformation was performed essentially as described (Wu et al. (1997) MPMI 10: 700-708). Briefly, M. grisea strain Guy 11 was grown in complete liquid media (Talbot et al. (1993) Plant Cell 5: 1575-1590 (PMID: 8312740)) shaking at 120 rpm for 3 days at 25° C. in the dark. Mycelia was harvested and washed with sterile H₂O and digested with 4 mg/ml beta-glucanase (InterSpex) for 4-6 hours to generate protoplasts. Protoplasts were collected by centrifugation and resuspended in 20% sucrose at the concentration of 2×10⁸ protoplasts/ml. 50 ul protoplast suspension was mixed with 10-20 ug of the cosmid DNA and pulsed using Gene Pulser II (BioRad) set with the following parameters: resistance 200 ohm, capacitance 25 uF, voltage 0.6 kV. Transformed protoplasts were regenerated in complete agar media (C M, Talbot et al. (1993) Plant Cell 5: 1575-1590 (PMID: 8312740)) with the addition of 20% sucrose for one day, then overlayed with CM agar media containing hygromycin B (250 ug/ml) to select transformants. Transformants were screened for homologous recombination events in the target gene by PCR (Hamer et al. (2001) Proc Natl Acad Sci USA 98: 5110-15 (PMID: 11296265)). Two independent strains were identified and are hereby referred to as KO1-5 and KO1-17, respectively. KO1-14 represents an ectopic transformant in which the transposon containing DNA fragment integrated at a nonhomologous site within the fungal genome and does not eliminate CHS2 activity.

### Example 6

Effect of Transposon Insertion on Magnaporthe Pathogenicity

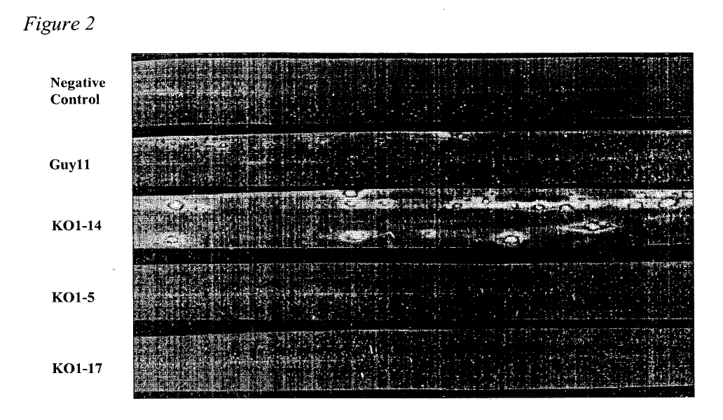

The fungal strains, KO1-5, KO1-17, and KO1-14 obtained in Example 5 and the wild type strain, Guy11, were subjected to a pathogenicity assay to observe infection over a 1-week period. Rice infection assays were performed using Indian rice cultivar CO39 essentially as described in Valent et al. ((1991) Genetics 127: 87-101 (PMID: 2016048)). All four strains were grown for spore production on complete agar media. Spores were harvested and the concentration of spores adjusted for whole plant inoculations. Two-week-old seedlings of cultivar CO39 were sprayed with 12 ml of conidial suspension (5×10⁴ conidia per ml in 0.01% Tween-20 (Polyoxyethylensorbitan monolaureate) solution). The inoculated plants were incubated in a dew chamber at 27° C. in the dark for 36 hours, and transferred to a growth chamber (27° C. 12 hours/21° C. 12 hours 70% humidity) for an additional 5.5 days. Leaf samples were taken at 3, 5, and 7 days post-inoculation and examined for signs of successful infection (i.e. lesions). FIG. 2 shows the effects of CHS2 gene disruption on Magnaporthe infection at five days post-inoculation.

### Example 7

Cloning and Expression Strategies, Extraction and Purification of Chitin Synthase 2 Protein.

The following protocol may be employed to obtain an isolated Chitin Synthase 2 protein.

Cloning and Expression Strategies:

A CHS2 cDNA gene can be cloned into E. coli (pET vectors-Novagen), Baculovirus (Pharmingen) and Yeast (Invitrogen) expression vectors containing His/fusion protein tags, and the expression of recombinant protein can be evaluated by SDS-PAGE and Western blot analysis.

Extraction:

Extract recombinant protein from 250 ml cell pellet in 3 ml of extraction buffer by sonicating 6 times, with 6 sec pulses at 4° C. Centrifuge extract at 15000×g for 10 min and collect supernatant. Assess biological activity of the recombinant protein by activity assay.

Purification:

Purify recombinant protein by Ni-NTA affinity chromatography (Qiagen).

Purification protocol: perform all steps at 4° C.:

Use 3 ml Ni-beads

Equilibrate column with the buffer

Load protein extract

Wash with the equilibration buffer

Elute bound protein with 0.5 M imidazole

### Example 8

Assays for Testing Binding of Test Compounds to Chitin Synthase 2

The following protocol may be employed to identify test compounds that bind to the Chitin Synthase 2 protein.

Isolated full-length Chitin Synthase 2 polypeptide with a His/fusion protein tag (Example 8) is bound to a HISGRAB Nickel Coated Plate (Pierce, Rockford, Ill.) following manufacturer's instructions.

Buffer conditions are optimized (e.g. ionic strength or pH, Cabib et al. (1987) Methods Enzymol 138: 643-9 (PMID: 2955198)) for binding of radiolabeled [¹⁴C]-Uridine Diphosphate N-Acetylglucosamine (American Radiolabeled Chemicals, Inc.) to the bound Chitin Synthase 2.

Screening of test compounds is performed by adding test compound and [¹⁴C]-Uridine Diphosphate N-Acetylglucosamine (American Radiolabeled Chemicals, Inc.) to the wells of the HISGRAB plate containing bound Chitin Synthase 2.

The wells are washed to remove excess labeled ligand and scintillation fluid (SCINTIVERSE, Fisher Scientific) is added to each well.

The plates are read in a microplate scintillation counter.

Candidate compounds are identified as wells with lower radioactivity as compared to control wells with no test compound added.

Additionally, an isolated polypeptide comprising 10-50 amino acids from the M. grisea Chitin Synthase 2 is screened in the same way. A polypeptide comprising 10-50 amino acids is generated by subcloning a portion of the CHS2 gene into a protein expression vector that adds a His-Tag when expressed (see Example 8). Oligonucleotide primers are designed to amplify a portion of the CHS2 gene using the polymerase chain reaction amplification method. The DNA fragment encoding a polypeptide of 10-50 amino acids is cloned into an expression vector, expressed in a host organism and isolated as described in Example 8 above.

Test compounds that bind CHS2 are further tested for antibiotic activity. M. grisea is grown as described for spore production on oatmeal agar media (Talbot et al. (1993) Plant Cell 5: 1575-1590 (PMID: 8312740)). Spores are harvested into minimal media to a concentration of 2×10⁵ spores/ml and the culture is divided. Id. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

### Example 9

Assays for Testing Inhibitors or Candidates for Inhibition of Chitin Synthase 2 Activity

The enzymatic activity of Chitin Synthase 2 is determined in the presence and absence of candidate compounds in a suitable reaction mixture, such as described by Cabib et al. (1987) Methods Enzymol 138: 643-9 (PMID: 2955198). Candidate compounds are identified when a decrease in products or a lack of decrease in substrates is detected with the reaction proceeding in either direction.

Additionally, the enzymatic activity of a polypeptide comprising 10-50 amino acids from the M. grisea Chitin Synthase 2 is determined in the presence and absence of candidate compounds in a suitable reaction mixture, such as described by Cabib et al. Id. A polypeptide comprising 10-50 amino acids is generated by subcloning a portion of the CHS2 gene into a protein expression vector that adds a His-Tag when expressed (see Example 8). Oligonucleotide primers are designed to amplify a portion of the CHS2 gene using polymerase chain reaction amplification method. The DNA fragment encoding a polypeptide of 10-50 amino acids is cloned into an expression vector, expressed and isolated as described in Example 8 above.

Test compounds identified as inhibitors of CHS2 activity are further tested for antibiotic activity. Magnaporthe grisea fungal cells are grown under standard fungal growth conditions that are well known and described in the art. M. grisea is grown as described for spore production on oatmeal agar media (Talbot et al. (1993) Plant Cell 5: 1575-1590 (PMID: 8312740)). Spores are harvested into minimal media to a concentration of 2×10⁵ spores/ml and the culture is divided. Id. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

### Example 10

Assays for Testing Compounds for Alteration of Chitin Synthase 2 Gene Expression

Magnaporthe grisea fungal cells are grown under standard fungal growth conditions that are well known and described in the art. Wild-type M. grisea spores are harvested from cultures grown on complete agar or oatmeal agar media after growth for 10-13 days in the light at 25° C. using a moistened cotton swab. The concentration of spores is determined using a hemacytometer and spore suspensions are prepared in a minimal growth medium to a concentration of 2×10⁵ spores per ml. 25 ml cultures are prepared to which test compounds will be added at various concentrations. A culture with no test compound present is included as a control. The cultures are incubated at 25° C. for 3 days after which test compound or solvent only control is added. The cultures are incubated an additional 18 hours. Fungal mycelia is harvested by filtration through MIRACLOTH (CalBiochem, La Jolla, Calif.), washed with water and frozen in liquid nitrogen. Total RNA is extracted with TRIZOL Reagent using the methods provided by the manufacturer (Life Technologies, Rockville, Md.). Expression is analyzed by Northern analysis of the RNA samples as described (Sambrook et al. (1989) Molecular Cloning, a Laboratory Manual, Cold Spring Harbor Laboratory Press) using a radiolabeled fragment of the CHS2 gene as a probe. Test compounds resulting in a reduced level of CHS2 mRNA relative to the untreated control sample are identified as candidate antibiotic compounds.

### Example 11

In Vivo Cell Based Assay Screening Protocol with a Fungal Strain Containing a Mutant Form of Chitin Synthase 2 with No Activity or Reduced Activity

Magnaporthe grisea fungal cells containing a mutant form of the CHS2 gene which abolishes enzyme activity, such as a gene containing a transposon insertion (see Examples 4 and 5), are grown under standard fungal growth conditions that are well known and described in the art. Magnaporthe grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild type cells are compared. Compounds that show differential growth inhibition between the mutant and the wild type are identified as potential antifungal compounds. Similar protocols may be found in Kirsch and DiDomenico ((1994) Biotechnology 26: 177-221 (PMID: 7749303)).

### Example 12

In Vivo Cell Based Assay Screening Protocol with a Fungal Strain Containing a Mutant Form of a Chitin Biosynthetic Gene with No Activity or Reduced Activity

Magnaporthe grisea fungal cells containing a mutant form of a gene in the chitin biosynthetic pathway (e.g. UTP:N-acetyl-alpha-D-glucosamine-1-phosphate uridylyltransferase (E.C. 2.7.7.23)) are grown under standard fungal growth conditions that are well known and described in the art. Magnaporthe grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. Wild type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild type cells are compared. Compounds that show differential growth inhibition between the mutant and the wild-type are identified as potential antifungal compounds. Similar protocols may be found in Kirsch and DiDomenico ((1994) Biotechnology 26: 177-221).

### Example 13

In Vivo Cell Based Assay Screening Protocol with a Fungal Strain Containing a Fungal CHS2 and a Second Fungal Strain Containing a Heterologous CHS2 Gene

Wild-type Magnaporthe grisea fungal cells and M. grisea fungal cells lacking a functional CHS2 gene and containing a class III chitin synthase G gene from Aspergillus fumigatus (Genbank 1353638, 67% sequence identity) are grown under standard fungal growth conditions that are well known and described in the art. A M. grisea strain carrying a heterologous CHS2 gene is made as follows: A M. grisea strain is made with a nonfunctional CHS2 gene, such as one containing a transposon insertion in the native gene (see Examples 4 and 5). A construct containing a heterologous CHS2 gene is made by cloning the class III chitin synthase G gene from Aspergillus fumigatus into a fungal expression vector containing a trpC promoter and terminator (e.g. pCB1003, Carroll et al. (1994) Fungal Gen News Lett 41: 22) using standard molecular biology techniques that are well known and described in the art (Sambrook et al. (1989) Molecular Cloning, a Laboratory Manual). The said construct is used to transform the M. grisea strain lacking a functional CHS2 gene (see Example 5). Transformants are selected on minimal agar medium lacking chitin. Only transformants carrying a functional CHS2 gene will grow.

Wild-type strains of Magnaporthe grisea and strains containing a heterologous form of CHS2 are grown under standard fungal growth conditions that are well known and described in the art. Magnaporthe grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590nm are taken daily. The effect of each compound on the wild-type and heterologous fungal strains is measured against the growth control and the percent of inhibition is calculated as the OD₅₉₀ (fungal strain plus test compound)/OD₅₉₀ (growth control)×100. The percent of growth inhibition as a result of a test compound on the wild-type and heterologous fungal strains are compared. Compounds that show differential growth inhibition between the wild-type and heterologous strains are identified as potential antifungal compounds with specificity to the native or heterologous CHS2 gene products. Similar protocols may be found in Kirsch and DiDomenico ((1994) Biotechnology 26: 177-221).

While the foregoing describes certain embodiments of the invention, it will be understood by those skilled in the art that variations and modifications may be made and still fall within the scope of the invention. The foregoing examples are intended to exemplify various specific embodiments of the invention and do not limit its scope in any manner.

What is claimed is:

1. A method for identifying a test compound as a candidate for an antibiotic, comprising:
a) contacting a Chitin Synthase 2 polypeptide with a test compound; and
b) detecting the presence or absence of binding between said test compound and said Chitin Synthase 2 polypeptide, wherein binding indicates that said test compound is a candidate for an antibiotic.

2. The method of claim 1, wherein said Chitin Synthase 2 polypeptide is a fungal Chitin Synthase 2 polypeptide.

3. The method of claim 1, wherein said Chitin Synthase 2 polypeptide is a Magnaporthe Chitin Synthase 2 polypeptide.

4. The method of claim 1, wherein said Chitin Synthase 2 polypeptide is SEQ ID NO: 3.

5. A method for determining whether the antibiotic candidate of claim 1 has antifungal activity, further comprising: contacting a fungus or fungal cells with said antibiotic candidate and detecting the decrease in growth, viability, or pathogenicity of said fungus or fungal cells.

6. A method for identifying a test compound as a candidate for an antibiotic, comprising:
a) contacting a test compound with at least one polypeptide selected from the group consisting of: a polypeptide having at least ten consecutive amino acids of a fungal Chitin Synthase 2; a polypeptide having at least 50% sequence identity with a fungal Chitin Synthase 2; and a polypeptide having at least 10% of the activity thereof; and
b) detecting the presence and/or absence of binding between said test compound and said polypeptide, wherein binding indicates that said test compound is a candidate for an antibiotic.

7. A method for determining whether the antibiotic candidate of claim 6 has antifungal activity, further comprising: contacting a fungus or fungal cells with said antibiotic candidate and detecting a decrease in growth, viability, or pathogenicity of said fungus or fungal cells.

8. A method for identifying a test compound as a candidate for an antibiotic, comprising:
a) contacting UDP-N-acetyl-D-glucosamine and starter chitin with a Chitin Synthase 2;
b) contacting UDP-N-acetyl-D-glucosamine and starter chitin with Chitin Synthase 2 and a test compound; and
c) determining the change in concentration for at least one of the following: UDP-N-acetyl-D-glucosamine, starter chitin, extended chitin, and/or UDP, wherein a change in concentration for any of the above substances between steps (a) and (b) indicates that said test compound is a candidate for an antibiotic.

9. The method of claim 8, wherein said Chitin Synthase 2 is a fungal Chitin Synthase 2.

10. The method of claim 8, wherein said Chitin Synthase 2 is a Magnaporthe Chitin Synthase 2.

11. The method of claim 8, wherein said Chitin Synthase 2 is SEQ ID NO: 3.

12. A method for determining whether the antibiotic candidate of claim 8 has antifungal activity, further comprising: contacting a fungus or fungal cells with said antibiotic candidate and detecting a decrease in growth, viability, or pathogenicity of said fungus or fungal cells.

13. A method for identifying a test compound as a candidate for an antibiotic, comprising:
a) contacting starter chitin and UDP with a Chitin Synthase 2;
b) contacting starter chitin and UDP with a Chitin Synthase 2 and a test compound; and
c) determining the change in concentration for at least one of the following: UDP-N-acetyl-D-glucosamine, starter chitin, shortened chitin, and/or UDP, wherein a change in concentration for any of the above substances between steps (a) and (b) indicates that said test compound is a candidate for an antibiotic.

14. The method of claim 13, wherein said Chitin Synthase 2 is a fungal Chitin Synthase 2.

15. The method of claim 13, wherein said Chitin Synthase 2 is a Magnaporthe Chitin Synthase 2.

16. The method of claim 13, wherein said Chitin Synthase 2 is SEQ ID NO: 3.

17. A method for determining whether the antibiotic candidate of claim 13 has antifungal activity, further comprising: contacting a fungus or fungal cells with said antibiotic candidate and detecting a decrease in growth, viability, or pathogenicity of said fungus or fungal cells.

18. A method for identifying a test compound as a candidate for an antibiotic, comprising:
a) contacting UDP-N-acetyl-D-glucosamine and starter chitin with a polypeptide selected from the group consisting of: a polypeptide having at least 50% sequence identity with Chitin Synthase 2; a polypeptide having at least 50% sequence identity with a Chitin Synthase 2 and having at least 10% of the activity thereof; and a polypeptide comprising at least 100 consecutive amino acids of a Chitin Synthase 2;
b) contacting UDP-N-acetyl-D-glucosamine and starter chitin with said polypeptide and a test compound; and
c) determining the change in concentration for at least one of the following: UDP-N-acetyl-D-glucosamine, starter chitin, extended chitin, and/or UDP, wherein a change in concentration for any of the above substances between steps (a) and (b) indicates that said test compound is a candidate for an antibiotic.

19. A method for identifying a test compound as a candidate for an antibiotic, comprising:
a) contacting starter chitin and UDP with a polypeptide selected from the group consisting of: a polypeptide having at least 50% sequence identity with a Chitin Synthase 2; a polypeptide having at least 50% sequence identity with a Chitin Synthase 2 and at least 10% of the activity thereof; and a polypeptide comprising at least 100 consecutive amino acids of a Chitin Synthase 2;
b) contacting starter chitin and UDP, with said polypeptide and a test compound; and
c) determining the change in concentration for at least one of the following: UDP-N-acetyl-D-glucosamine, starter chitin, shortened chitin, and/or UDP, wherein a change in concentration for any of the above substances between steps (a) and (b) indicates that said test compound is a candidate for an antibiotic.

20. A method for identifying a test compound as a candidate for an antibiotic, comprising:
a) measuring the expression of a Chitin Synthase 2 in a cell, cells, tissue, or an organism in the absence of a test compound;
b) contacting said cell, cells, tissue, or organism with said test compound and measuring the expression of said Chitin Synthase 2 in said cell, cells, tissue, or organism; and
c) comparing the expression of Chitin Synthase 2 in steps (a) and (b), wherein a lower expression in the presence of said test compound indicates that said test compound is a candidate for an antibiotic.

21. The method of claim 20, wherein said cell, cells, tissue, or organism is, or is derived from a fungus.

22. The method of claim 20, wherein said cell, cells, tissue, or organism is, or is derived from a Magnaporthe fungus or fungal cell.

23. The method of claim 20, wherein said Chitin Synthase 2 is SEQ ID NO: 3.

24. The method of claim 20, wherein the expression of Chitin Synthase 2 is measured by detecting CHS2 mRNA.

25. The method of claim 20, wherein the expression of Chitin Synthase 2 is measured by detecting Chitin Synthase 2 polypeptide.

26. A method for identifying a test compound as a candidate for an antibiotic, comprising:
a) providing cells having one form of a Chitin Synthase 2 gene, and providing comparison cells having a different form of a Chitin Synthase 2 gene; and
b) contacting said cells and said comparison cells with a test compound and determining the growth of said cells and comparison cells in the presence of the test compound, wherein a difference in growth between said cells and said comparison cells in the presence of said compound indicates that said compound is a candidate for an antibiotic.

27. The method of claim 26, wherein the cells and the comparison cells are fungal cells.

28. The method of claim 26, wherein the cells and the comparison cells are Magnaporthe cells.

29. The method of claim 26, wherein said form and said different form of the Chitin Synthase 2 are fungal Chitin Synthase 2s.

30. The method of claim 26, wherein at least one of the forms is a Magnaporthe Chitin Synthase 2.

31. The method of claim 26, wherein said form and said different form of the Chitin Synthase 2 are non-fungal Chitin Synthase 2s.

32. The method of claim 26, wherein one form of the Chitin Synthase 2 is a fungal Chitin Synthase 2, and the different form is a non-fungal Chitin Synthase 2.

33. A method for identifying a test compound as a candidate for an antibiotic, comprising:
a) providing cells having one form of a gene in the chitin biochemical and/or genetic pathway and providing comparison cells having a different form of said gene.
b) contacting said cells and said comparison cells with a said test compound,
c) determining the growth of said cells and said comparison cells in the presence of said test compound, wherein a difference in growth between said cells and said comparison cells in the presence of said test compound indicates that said test compound is a candidate for an antibiotic.

34. The method of claim 33, wherein the cells and the comparison cells are fungal cells.

35. The method of claim 33, wherein the cells and the comparison cells are Magnaporthe cells.

36. The method of claim 33, wherein said form and said different form of the chitin biosynthesis gene are fungal chitin biosynthesis genes.

37. The method of claim 33, wherein at least one of the forms is a Magnaporthe chitin biosynthesis gene.

38. The method of claim 33, wherein said form and said different form of the chitin biosynthesis genes are non-fungal chitin biosynthesis genes.

39. The method of claim 33, wherein one form of the chitin biosynthesis gene is a fungal chitin biosynthesis gene, and the different form is a non-fungal chitin biosynthesis gene.

40. A method for determining whether the antibiotic candidate of claim 33 has antifungal activity, further comprising: contacting a fungus or fungal cells with said antibiotic candidate and detecting a decrease in growth, viability, or pathogenicity of said fungus or fungal cells, wherein a decrease in growth, viability, or pathogenicity of said fungus or fungal cells indicates that the antibiotic candidate has antifungal activity.

41. An isolated nucleic acid comprising a nucleotide sequence that encodes a polypeptide of SEQ ID NO: 3.

42. The nucleic acid of claim 41 comprising the nucleotide sequence of SEQ ID NO: 1.

43. An expression cassette comprising the nucleic acid of claim 42.

44. The isolated nucleic acid of claim 41 comprising a nucleotide sequence with at least 50 to at least 95% sequence identity to SEQ ID NO: 1.

45. An isolated polypeptide consisting essentially of the amino acid sequence of SEQ ID NO: 3.

46. An isolated polypeptide comprising the amino acid sequence of SEQ ID NO: 3.
